(19) United States
(12) Patent Application Publication
(10) Pub. No.: US 2025/0128242 A1
(43) Pub. Date: Apr. 24, 2025

(54) METHOD AND CATALYST FOR METHANE CONVERSION TO CYCLOHEXANE

(71) Applicant: THE ROYAL INSTITUTION FOR THE ADVANCEMENT OF LEARNING/MCGILL UNIVERSITY, Montréal (CA)

(72) Inventors: Chao-Jun LI, Brossard (CA); Lida TAN, Montréal (CA)

(21) Appl. No.: 18/684,998

(22) PCT Filed: Aug. 31, 2022
(86) PCT No.: PCT/CA2022/051315

§ 371 Date: Feb. 20, 2024

Related U.S. Application Data
(60) Provisional application No. 63/240,048, filed on Sep. 2, 2021.

(51) Int. Cl. B01J 23/62; B01J 27/24; B01J 35/30; B01J 37/02; B01J 37/06; B01J 37/08; B01J 37/16; B01J 37/34; C07C 2/84

(52) U.S. Cl. CPC B01J 23/62; B01J 27/24; B01J 35/393; B01J 37/0201; B01J 37/06; B01J 37/08; B01J 37/16; B01J 37/341; C07C 2/84; C07C 2523/42; C07C 2527/24

(57) ABSTRACT

There is provided a catalyst for the conversion of methane to cyclohexane. The catalyst comprises gallium nitride, zinc oxide, gallium oxide or a combination thereof; and platinum clusters deposited at the surface of the gallium nitride, the zinc oxide, the gallium oxide or the combination thereof. The platinum clusters collectively represent from about 0.75 to about 4% by weight of the catalyst.

### CROSS-REFERENCE TO RELATED APPLICATIONS

The present application claims priority from U.S. provisional patent application 63/240,048 filed on Sep. 2, 2021 and herewith incorporated by reference in its entirety.

### TECHNICAL FIELD

This disclosure relates to the field of catalytic cyclohexane production from methane.

### BACKGROUND OF THE ART

As petroleum resources deplete over time, the numerous reserves of methane may replace petroleum as an alternative carbon-based feedstock. However, despite the abundancy of methane, the reserves of methane are mainly located in remote locations where large pipeline constructions need to be built to transport methane to marketplace. Therefore, the liquefaction of methane is highly desirable to facilitate the transport of methane. Methane has been converted into certain liquefied products such as methanol, acetic acid and aromatics (BTX). Particularly, the conversion of methane to benzene has been demonstrated in Li, L., Mu, X., Liu, W., Kong, X., Fan, S., Mi, Z., & Li, C. J. (2014). Thermal Non-Oxidative Aromatization of Light Alkanes Catalyzed by Gallium Nitride. Angewandte Chemie, 126(51), 14330-14333. Cyclohexane is a valuable liquid as a petroleum alternative. For example, cyclohexane is an important intermediate in the synthesis of Nylon. The conventional production of cyclohexane is relied on the hydrogenation of benzene, which is heavily based on the petroleum industry. An example, of benzene conversion to cyclohexane is described in Li, L., Mu, X., Liu, W., Mi, Z., & Li, C. J. (2015). Simple and efficient system for combined solar energy harvesting and reversible hydrogen storage. Journal of the American Chemical Society, 137(24), 7576-7579. However, methods and catalysts for the direct liquefaction of methane to cyclohexane are desirable.

### SUMMARY

In one aspect, there is provided a catalyst for the conversion of methane to cyclohexane, the catalyst comprising: gallium nitride, zinc oxide, gallium oxide or a combination thereof; and platinum clusters deposited at the surface of the gallium nitride, the zinc oxide, the gallium oxide or the combination thereof, wherein the platinum clusters collectively represent from about 0.75 to about 4% by weight of the catalyst. In some embodiments, the catalyst comprises at least 95% by weight of gallium nitride. In such embodiments, the GaN catalyst may have a platinum 4f_{7/2} binding energy peak of less than 74.0 eV determined by X-ray photoelectron spectroscopy and/or a platinum 4f_{5/2} binding energy peak of less than 71.0 eV determined by X-ray photoelectron spectroscopy. In further embodiments, the platinum clusters collectively are from about 0.75 to about 2% by weight of the catalyst. The catalyst may be a nanoparticle.

In a further aspect, there is provided a method of producing cyclohexane comprising the step of: contacting the catalyst of the present disclosure and methane in a reaction vessel at a temperature of from about 250° C. to about 350° C. for a time sufficient to produce the cyclohexane. The sufficient time can be between 1 to 3 hours. The catalyst and the methane may be contacted under vacuum or an in inert atmosphere. In some embodiments, the method further comprises drying the methane before the step of contacting the methane with the catalyst. In additional embodiments, the method further comprises cleaning the catalyst before the step of contacting the methane with the catalyst. Cleaning may include purging the catalyst with methane.

In yet a further aspect, there is provided a process of making the catalyst of the present disclosure, the process comprising the step of: depositing from about 0.75 to about 4% by weight of platinum on the surface of the gallium nitride (such as a GaN nanoparticle), the zinc oxide, the gallium oxide or the combination thereof to obtain the catalyst by chemical reduction, such as a photodeposition. In some embodiments, the process further comprises washing the catalyst after the step of depositing. In yet further embodiments, the process further comprises drying the catalyst after the step of washing.

Many further features and combinations thereof concerning the present improvements will appear to those skilled in the art following a reading of the instant disclosure.

### DESCRIPTION OF THE DRAWINGS

FIG. 1 is a schematic representation of a catalyst according to an embodiment of the present disclosure.

FIG. 2 is a schematic representation of a photoreduction process of Pt/GaN catalyst according to an embodiment of the present disclosure.

FIG. 3A is a transmission electron microscopy (TEM) image of an exemplary catalyst according to the present disclosure.

FIG. 3B is a high-angle annular dark-field imaging scanning transmission electron microscopy (HAADF-STEM) image of 1 wt % Pt/GaN.

FIG. 3C is the corresponding energy-dispersive X-ray (EDX) maps of the HAADF-STEM image of FIG. 2B.

FIG. 3D is the corresponding EDX maps of HAADF-STEM of FIG. 2B showing Ga atoms.

FIG. 3E is the corresponding EDX maps of HAADF-STEM of FIG. 2B showing N atoms.

FIG. 3F is the corresponding EDX maps of HAADF-STEM of FIG. 2B showing Pt atoms.

FIG. 3G is a HAADF-STEM images of 1 wt % Pt/GaN according to an exemplary embodiment.

FIG. 3H is the EDX spectrum of FIG. 2G, showing the energy (kev) as a function of intensity (kCounts).

FIG. 3I is a HAADF-STEM image of Pt₂/GaN.

FIG. 3J is a close up of the HAADF-STEM image of Pt₂/GaN of FIG. 3I (dotted rectangle zone in FIG. 3I).

FIG. 3K is a HAADF-STEM image of Pt₄/GaN.

FIG. 3L is a close up of the HAADF-STEM image of Pt₄/GaN of FIG. 3K (dotted rectangle zone in FIG. 3K).

FIG. 4A is a Pt 4f XPS spectra of Pt/GaN and K₂PtCl₄.

FIG. 4B is a full XPS spectra of Pt_{0.5}/GaN, Pt₁/GaN, Pt₂/GaN, and Pt₄/GaN samples.

FIG. 4C is a Ga 3d XPS spectra of the Pt_{0.5}/GaN, Pt₁/GaN, Pt₂/GaN, and Pt₄/GaN samples of FIG. 4B.

FIG. 4D is a Ga 3d XPS spectra of the Pt_{0.5}/GaN, Pt₁/GaN, Pt₂/GaN, and Pt₄/GaN samples of FIG. 4B (dotted rectangle zone in FIG. 4B).

FIG. 4E is a powder X-ray diffraction (XRD) pattern of Pt/GaN and GaN.

FIG. 4F is a plot of Brunauer-Emmett-Teller (BET) surface area measurement of GaN.

FIG. 4G is a plot of BET surface area measurement of Pt/GaN.

FIG. 4H is a microscopy image of a Pt/GaN sample.

FIG. 4I is a graph showing the frequency in function of particle diameter.

FIG. 5 is the X-ray photoelectron spectroscopy (XPS) spectra of an exemplary catalyst according to the present disclosure.

FIG. 6A is a bar graph of the conversion and distribution of products for 1 wt % Pt/GaN at different reaction temperatures according to exemplary embodiments. The reaction was performed at 300° C. for 2 hours.

FIG. 6B is a bar graph of the conversion and distribution of products with various Pt loading in the Pt/GaN exemplary catalyst. The reaction was performed at 300° C. for 2 hours.

FIG. 7A is a bar graph of the conversion and distribution of products obtained with the exemplary catalyst 1 wt % Pt/GaN after 5 cycles.

FIG. 7B is the XPS spectra of the catalyst of FIG. 5A before any reaction and after the 5 cycles of reaction.

FIG. 8A is a graph showing the yield of benzene with the catalysts TiO₂, Ga₂O₃, ZnO and GaN.

FIG. 8B is a gas chromatography—thermal conductivity detector (GC-TCD) spectra of the gas sample obtained by Pt₁/GaN at 300° C. after 2 h.

FIG. 8C is a GC-TCD spectra of cyclohexane, standard hydrocarbon mixture (C1-C6) and the gas sample obtained by Pt₁/GaN as per FIG. 8B.

FIG. 8D is a graph showing the yield of cyclohexane over various catalyst supports with and without loading Pt.

FIG. 8E is a graph showing the selectivity and productivity of methane transformation by Ptₓ/GaN catalysts (x=0.5, 1, 2, or 4 wt. %).

FIG. 8F graph showing the catalytic performance Pt₁/GaN at various reaction temperatures.

FIG. 9A is a graph showing the yield of cyclohexane and benzene (A=benzene, B=cyclohexane) over time with the mixture of Pt/C and GaN.

FIG. 9B is a graph showing the yield of cyclohexane and benzene (A=benzene, B=cyclohexane) over time with the mixture of Pt/GaN.

FIG. 9C is a schematic illustration of benzene reacting with hydrogen gas over the Pt/GaN catalyst.

FIG. 9D is a gas chromatography-mass spectrometry (GCMS) spectra (intensity in function of retention time) for the gas sample after the hydrogenation of benzene over Pt₁/GaN at 300° C. with the reaction conditions: 20 mg of catalyst, 4.5 μmol of benzene, 27 μmol of H₂, 2 h.

FIG. 9E is a gas chromatography-mass spectrometry (GCMS) spectra (intensity in function of molecular weight) for the gas sample after the hydrogenation of benzene over Pt₁/GaN at 300° C. with the reaction conditions: 20 mg of catalyst, 4.5 μmol of benzene, 27 μmol of H₂, 2 h.

FIG. 9F is a schematic illustration of an exemplary methane conversion pathway for the production of cyclohexane catalyzed by Pt/GaN interface.

FIG. 10A is a graph showing the yield of cyclohexane over reused Pt/GaN with the reaction conditions: 2 mmol of methane, 20 mg of catalyst, 300° C., 2 h.

FIG. 10B is a Raman spectra of Pt₁/GaN before (“fresh”) and after (“used”) reaction.

FIG. 10C is a bar graph showing the carbon element content on the catalyst surface of GaN and Pt₁/GaN before and after reaction.

FIG. 10D is a GC-MS chromatography spectra (intensity in function of reaction time) for the gas sample over Pt₁/GaN at 300° C. with the reaction conditions: 20 mg of catalyst, 1 atm, 50 mL of reagent gas, 2 h, in Ar gas.

FIG. 10E is a GC-MS chromatography spectra (intensity in function of reaction time) for the gas sample over Pt₁/GaN at 300° C. with the reaction conditions: 20 mg of catalyst, 1 atm, 50 mL of reagent gas, 2 h, in ¹²C methane gas.

FIG. 10F is a GC-MS chromatography spectra (intensity in function of molecular weight) for the gas sample over Pt₁/GaN at 300° C. with the reaction conditions: 20 mg of catalyst, 1 atm, 50 mL of reagent gas, 2 h, in ¹²C methane gas.

FIG. 10G is a close up of FIG. 10F around peak 84.

FIG. 10H is a GC-MS chromatography spectra (intensity in function of reaction time) for the gas sample over Pt₁/GaN at 300° C. with the reaction conditions: 20 mg of catalyst, 1 atm, 50 mL of reagent gas, 2 h, in a 50:50 mixture of ¹²C and ¹³C methane gas.

FIG. 10I is a GC-MS chromatography spectra (intensity in function of molecular weight) for the gas sample over Pt₁/GaN at 300° C. with the reaction conditions: 20 mg of catalyst, 1 atm, 50 mL of reagent gas, 2 h, in a 50:50 mixture of ¹²C and ¹³C methane gas.

FIG. 10J is Pt 4f XPS spectra of fresh and used Pt₁/GaN.

FIG. 11A is a scanning electron microscopy (SEM) of fresh Pt₁/GaN.

FIG. 11B is a close up of FIG. 11A at the dotted rectangle.

FIG. 11C is a SEM of used Pt₁/GaN.

FIG. 11D is a close up of FIG. 11C at the dotted rectangle.

FIG. 12A is a HAADF-STEM image of used Pt₁/GaN (i.e. after reaction).

FIG. 12B is an EDX mapping of Pt in the used catalyst of FIG. 12A.

FIG. 12C is an EDX mapping of Ga in the used catalyst of FIG. 12A.

FIG. 12D is an EDX mapping of N in the used catalyst of FIG. 12A.

FIG. 13 is a XRD spectroscopy of Pt₁/GaN before and after reaction.

FIG. 14 is a graph of the calibration curve obtained for hydrogen gas quantification.

### DETAILED DESCRIPTION

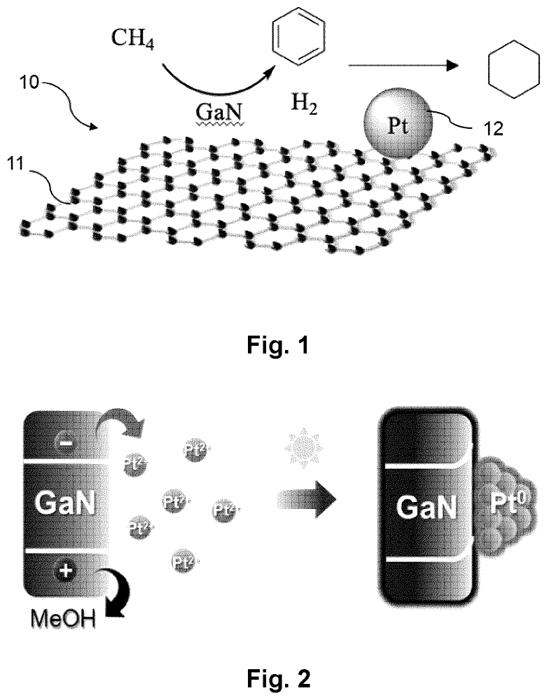

The main challenge for the methane transformation to cyclohexane is the inertness of C—H bonds which require a large activation energy. The inventors of the present disclosure, have surprisingly found a catalyst that allows for methane activation under thermal conditions to generate cyclohexane. The catalyst of the present disclosure comprises i) gallium nitride (GaN), zinc oxide (ZnO) or gallium oxide (Ga₂O₃), and ii) platinum (Pt) clusters distributed on a surface of the GaN, ZnO, or Ga₂O₃ where the platinum clusters collectively are from about 0.75 to about 4%, from about 1 to about 4%, from about 0.75 to about 2%, from about 0.75 to about 1.5%, from about 0.75 to about 1.25%, or about 1% by weight of the catalyst. The term “about” as used herein in the context of a weight percentage is defined as ±20%, 15%, 10%, 5%, or ±3%. GaN, ZnO and Ga₂O₃ catalyze the formation of benzene from methane and Pt catalyzes the conversion of benzene to cyclohexane. The term “platinum clusters” or “Pt clusters” as used herein refers to clusters of Pt atoms containing at least one Pt atom and/or having a diameter of less than 2 nm deposited on the catalyst surface (i.e. a surface of GaN). In one example, a Pt cluster comprises or consists of 1 to 10 Pt atoms. In one embodiment, the Pt atoms forming a Pt cluster are bonded together with metal-metal bonds. Making reference to FIG. 1, a catalyst 10 according to an embodiment comprises GaN 11 and a Pt cluster 12. More specifically an atomic 2D planar schematic is shown for GaN 11 and a Pt cluster 12 being in this exemplary case a single Pt atom, is deposited onto the GaN 11. The Pt atom is linked to the GaN atomic structure by a covalent chemical linkage between N and Pt.

In some embodiments, the catalyst comprises at least 90%, at least 93%, at least 95%, at least 96% or at least 97% by weight of GaN, ZnO, Ga₂O₃ or combinations thereof, with respect to the total weight of the catalyst. GaN is an III-V nitride semiconductor with a d¹⁰ electronic configuration. The very high bonding energy (8.9 eV/atom) of the GaN bond with a largely ionic component character, makes GaN a thermally and chemically stable material with an ultrahigh melting point (>2500° C.). GaN is resistant to decomposition at least up to 1000° C., even under vacuum.

In one example, the catalyst is a nanoparticle such as a GaN nanoparticle. In one embodiment, GaN nanoparticles are characterized by a regular wurtzite crystal structure, which is the thermodynamically stable phase of GaN. In this crystal structure, the exposed surfaces of the GaN nanoparticles are composed of c-planes and m-planes. The m-plane is a one dimensional rectangular configuration of the Ga and N atoms and the c-plane is a one dimensional hexagonal configuration of the Ga and N atoms. The overall m-plane of GaN is nonpolar since it is composed of equal numbers of Ga and N atoms which are tetrahedrally coordinated with each other, whereas the polar c-plane comprises only one type of atom (either Ga or N) which exhibits piezoelectric polarization along the c-axis. The term “nanoparticle” as used herein may be defined as having a diameter in the nanoscale. For example, the GaN nanoparticles can have a diameter of between 10 and 1000 nm, between 20 and 900 nm, between 30 and 800 nm, or between 40 and 700 nm.

In some embodiments, the platinum clusters are present in a concentration in weight percent with respect to the total weight of the catalyst of from 0.75 to 4%, from 1 to 4%, from 0.75 to 2%, from 0.75 to 1.5% or from 0.75 to 1.25% by weight of the catalyst. The platinum clusters may be randomly dispersed on a surface of the catalyst. In one embodiment, the Pt clusters are spread out on the surface of the catalyst such that the Pt clusters are sporadically present across all the surface area. In one example, the platinum clusters can be distributed on more than one surface. Without wishing to be bound by theory, a smaller platinum cluster is believed to produce a better catalytic activity. In one embodiment, platinum clusters are separated by at least 4 nm on the surface of the catalyst.

At industrial scale, the catalyst may be an unsupported or a supported sheet in a reactor, such as a flow reactor. At industrial scale, when a continuous flow of methane is supplied the concentration of Pt clusters can be modified based on the flow rate. For example, when the flow rate is high the concentration of Pt clusters can be reduced so as to space the clusters across a longer distance which is covered in a shorter period of time because of the high flow rate. Accordingly, although a small yield is obtained at laboratory scale for a small concentration of Pt clusters, this does not mean that such a concentration is not viable at industry scale, particularly in flow reactors. In some embodiments, the catalyst is suspended in a reactor or is deposited onto a reactor bed or surface. In additional embodiments, the catalyst itself may be deposited onto at least a portion of a reaction surface (i.e. support by the reactor). In further embodiments, industrial scale processes may be performed at a pressure higher than, or equal to, 1 atm, for example 2 atm or more.

In some embodiments, the catalyst of the present disclosure is a GaN catalyst with Pt clusters and has a catalysis activity for the direct conversion of methane to cyclohexane according to the scheme below.

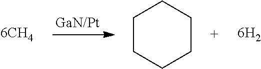

In some embodiments, the catalyst is a GaN catalyst characterized by X-ray photoelectron spectroscopy (XPS). In one embodiment, the GaN catalyst has a platinum 4f₇/2 binding energy peak of less than 74.0 eV, less than 73.9 eV, less than 73.8 eV, less than 73.7 eV, less than 73.6 eV, or less than 73.5 eV. In one example, the platinum 4f_{7/2} binding energy peak is between 73.1 and 73.5 eV. In a further embodiment, the catalyst has a platinum 4f₅/2 binding energy peak of less than 71.0 eV, less than 70.9 eV, less than 70.8 eV, less than 70.7 eV, less than 70.6 eV, less than 70.5 eV, or less than 70.4 eV. In one example, the platinum 4f₅/2 binding energy peak is between 70.0 and 70.4 eV. The XPS can for example be operated with a monochromated X-ray source (Al kα hv=1486.6 eV) and the energy scale can be calibrated using Au 4f₇/2, Cu 2p₃/2, and Ag 3d_{5/2} peak positions. Without wishing to be bound by theory, the peak shift for the binding energy of Pt 4f_{7/2} and 4f_{5/2} from the initial 74.8 and 71.2 eV respectively, is believed to improve the catalytic activity of the formation of cyclohexane because a higher electron density is beneficial to the activation of H₂ for the hydrogenation of benzene. Surprisingly, the present inventors have found through kinetic studies that it is the hydrogen species generated during the cleavage of the C—H bonds that participate in the hydrogenation of benzene instead of the hydrogen gas. Accordingly, without wishing to be bound by theory, the inert C—H bonds of methane are weakened by the electrostatic stretch induced by gallium and nitrogen atoms on the surface of GaN. After the cleavage of C—H bonds, —CHₓ species dimerize to C₂H_{y}, which undergo oligomerization and cyclization to benzene. Hydrogen species attached to the nitrogen atoms spill over towards the Pt cluster and reduce benzene. The high electron density of Pt facilitates this hydrogen transfer process.

Accordingly, there is provided a method of producing cyclohexane by contacting the catalyst according to the present disclosure with methane in a reaction vessel at a temperature of from about 250° C. to about 350° C., from 250° C. to 350° C., from about 250° C. to about 300° C. or about 300° C. to produce the cyclohexane. The term “about” as used herein in the context of a temperature value means ±20%, 15%, 10%, 5%, ±3%. Alternatively, the term “about” as used herein in the context of a temperature value means ±50° C., ±40° C., ±30° C., ±20° C., 15° C., ±10° C., or ±5° C. The present disclosure, demonstrated that benzene is readily hydrogenated to cyclohexane by hydrogen species with the Pt clusters of the catalyst under the same conditions as the formation of the benzene. Therefore, the present disclosure provides a direct conversion from methane to cyclohexane with high selectivity with the Pt/GaN catalyst of the present disclosure.

The method of producing cyclohexane according to the present disclosure, in one example, achieves a selectivity of at least 35, 38, 40, 50, 60, 65, 70, 75, 80, 85, or 90% selectivity for cyclohexane. The % selectivity is defined herein as the weight percentage of cyclohexane within the total products produced by the reaction.

Moreover, the method of producing cyclohexane according to the present disclosure optionally further comprises drying the methane before the reaction. The drying step can be performed by passing the gas through a drying column. After the cyclohexane is obtained, the catalyst can optionally be recycled and reused. In one embodiment to recycle the catalyst, a used catalyst is subjected to a thermal treatment at 450° C.-550° C. under vacuum for 2-4 hours to remove the remaining reactant and products. The catalyst can alternatively or additionally be subject to a washing step followed by a drying step. In one embodiment, the catalyst is purged with methane. In particular embodiments where the process is performed continuously, the catalyst is continuously purged with methane.

There is also provided a process for fabricating the catalyst according to the present disclosure. A gallium nitride material, a zinc oxide material or a gallium oxide material is provided. In one example, the materials are purchased commercially or are synthesized. The GaN material can be GaN nanoparticles. The synthesis of GaN and GaN nanoparticles can be performed with any suitable synthesis method. For example, a Ga precursor (e.g. elementary Ga or Ga₂O₃) can be reacted with a N precursor (e.g. NH₃ or N₂) at low pressure (e.g. less than 100 atm) and high temperature (e.g. more than 750° C.) to form GaN. In further embodiments, the synthesis is performed under vacuum or in an inert atmosphere. The term “vacuum” as defined herein refers to a pressure of less than 1 atm. The term “inert atmosphere” refers to atmosphere that comprises essentially or consists of non-reactive species that do not interfere with the reaction and produce secondary products. In one example, the inert atmosphere is or comprises Ar, He, or Ne. GaN may be synthesized by molecular beam epitaxy, metalorganic vapour phase epitaxy, or plasma techniques. To obtain the catalyst of the present disclosure from about 0.75 to about 4%, from 1 about to about 4%, from about 0.75 to about 2%, from about 0.75 to about 1.5%, from about 0.75 to about 1.25% or about 1% by weight of the catalyst by weight of platinum is deposited on a surface of the GaN, ZnO, or Ga₂O₃ material to form platinum clusters. In one embodiment, the deposition is a chemical reduction, for example a photodeposition. An exemplary photodeposition can be: (i) dissolving a Pt precursor (e.g. K₂PtCl₄) in a solvent comprising dionized water and methanol (e.g. in a ratio of 3:1 respectively); (ii) adding the GaN to the solution comprising Pt; (iii) photoirradiating the solution for, for example, 3 h; (iv) recovering the catalyst (GaN material with Pt fixed on its surface); and (v) performing one or more washing cycles. Washing the catalyst can be done using distilled water, methanol and/or combinations thereof. A washing cycling can include drying the catalyst. For example, the catalyst can be dried under vacuum at 100° C. for an extended period of time (e.g. overnight). Other suitable deposition techniques can be applied, such as for example those described in Wenderich, K., & Mul, G. (2016). Methods, mechanism, and applications of photodeposition in photocatalysis: a review. Chemical reviews, 116(23), 14587-14619 which is incorporated herein by reference in its entirety.

### Example 1: Fabrication of the Catalyst

Commercial GaN catalyst (99.9% purity) was purchased from Sigma-Aldrich and used without further treatment. Methane (99.99% purity) was purchased from Air Liquide. The metal precursor (K₂PtCl₄) and other catalyst supports (Ga₂O₃, TiO₂, Al₂O₃, C₃N₄) are commercially available compounds which were purchased from Sigma-Aldrich and used without further purification.

Pt/GaN was prepared based by a photodeposition method. In the typical synthesis of of Ptₓ/GaN (x=0.5%, 1%, 2% and 4%), K₂PtCl₄ (0.50 mg, 1.00 mg, 2.00 mg, and 4.00 mg) was dissolved in deionized (DI) water (6.00 mL) and methanol (2.00 mL) as stock solution. 20 mg of the GaN nanoparticles was dispersed in 4 mL of the stock solution in a quartz tube and was stirred under photoirradiation of xenon lamp (PE300 BUV) for 3 hours in argon gas. The suspension was collected by centrifugation and was washed with distilled water and methanol for three times. The final sample was gained after drying under vacuum at 300° C. for 1 hour and 100° C. overnight.

The photodeposition method was a photochemical synthesis strategy in which methanol was used as photogenerated hole scavenger, enabling more photogenerated electrons to reduce the Pt precursor (K₂PtCl₄) to metallic Pt (FIG. 2).

The deposition of Pt on other catalysts supports (Ga₂O₃, TiO₂, Al₂O₃) was based on the photodeposition method described in (Snyder, B. E. R. et al. Cage effects control the mechanism of methane hydroxylation in zeolites. Science 373, 327-331 (2021)). During the preparation, 1.00 mg K₂PtCl₄ was dissolved in deionized water (6.00 mL) and methanol (2.00 mL) as stock solution. 20 mg of the catalyst supports were dispersed in 4 mL of the stock solution in a quartz tube and was stirred under photoirradiation for 3 hours under a xenon lamp (PE300 BUV) in argon environment. The suspension was collected by centrifuge and was washed with distilled water and methanol several times and was dried under vacuum at 300° C. for 1 hour and 100° C. overnight.

### Example 2: Characterization of the Catalyst

High resolution bright field transmission electron microscope (TEM) images were obtained using FEl Tecnai™ G2 F20 S/TEM at accelerating voltage of 200 kV. For high-angle annular dark-field imaging scanning transmission electron microscopy (STEM-HAADF or HAADF-STEM) imaging, a Hitachi™ HD2700 Cs-corrected STEM was used with a cold field emitter operated at 200 kV and with an electron beam diameter of ˜0.1 nm. STEM energy-dispersive X-ray spectroscopy (EDXS) analysis was performed using a 60 mm² silicon drift detector from Bruker™. The X-ray photoelectron spectroscopy (XPS) was performed on an ESCALAB™ 250 X-ray photoelectron spectrometer with a monochromated X-ray source (Al kα hv=1486.6 eV), and the energy calibration of the spectrometer was performed using C 1s peak at 284.8 eV. The energy scale of the spectrometer was calibrated using Au 4f_{7/2}, Cu 2p_{3/2}, and Ag 3d_{5/2} peak positions. The scanning electron microscopy (SEM) was carried out on a FEl Quanta 450 Environment scanning electron microscopy (FE-ESEM) with EDAX™ Octane Super 60 mm² SDD and TEAM EDS Analysis system. The powder X-ray diffraction (XRD) patterns were obtained on a Bruker™ DD8 Advanced diffractometer with Cu Kα radiation (λ=1.5418 Å). Raman spectroscopy was performed on Confocal Raman Microscope (Alpha300, Witec, Ulm, Germany) with a piezo scanner (P-500, Physik Instrumente, Karlsruhe, Germany). The scattered light was measured via a thermoelectrically cooled CCD detector (DU401A-BV, Andor, Belfast, North Ireland) behind the spectrometer (UHTS 300, WITec, Ulm, Germany).

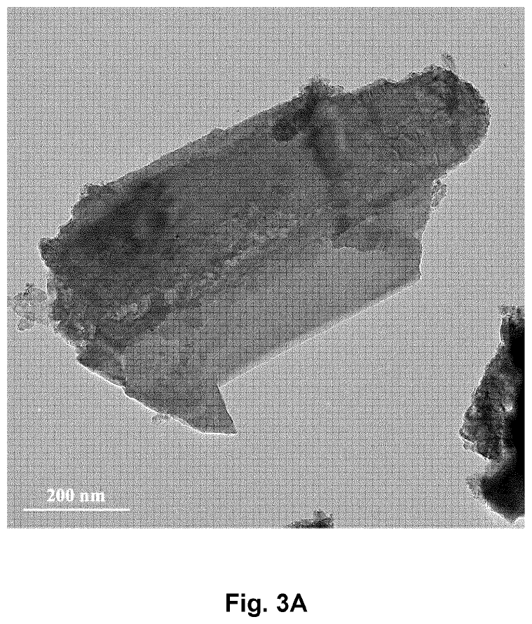

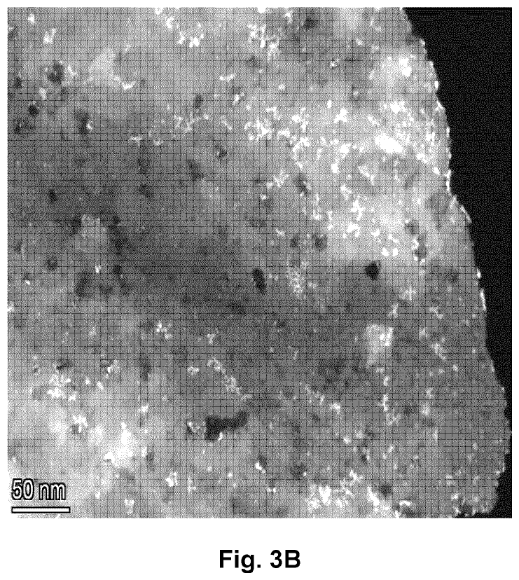

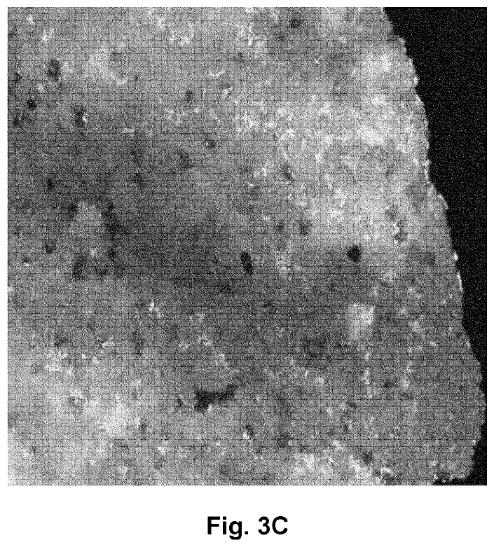

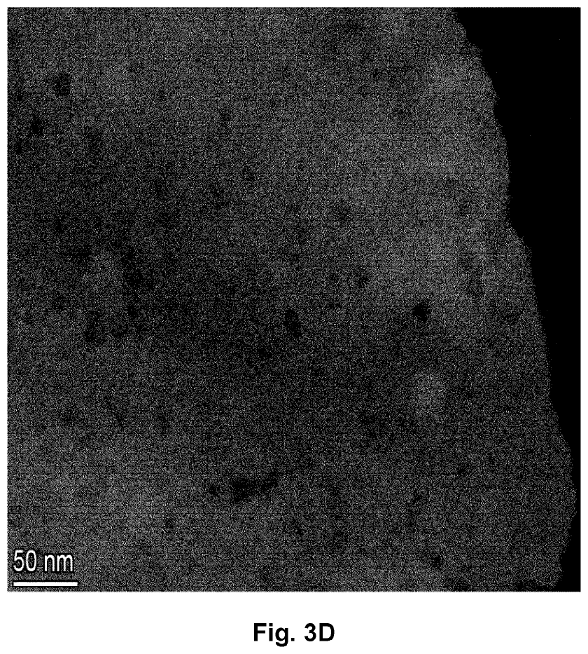

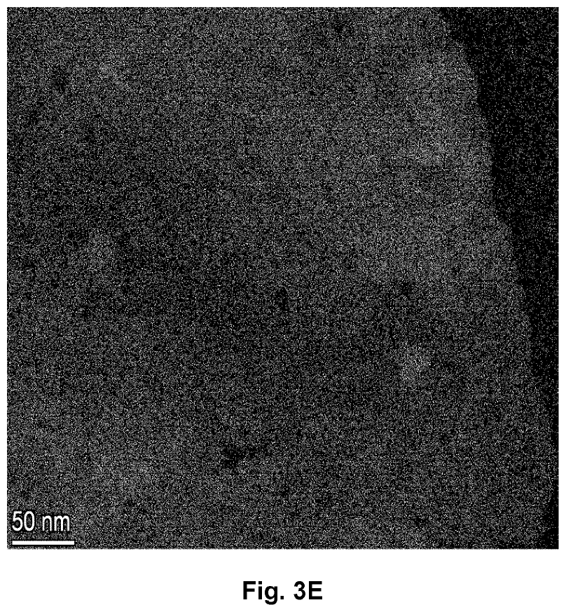

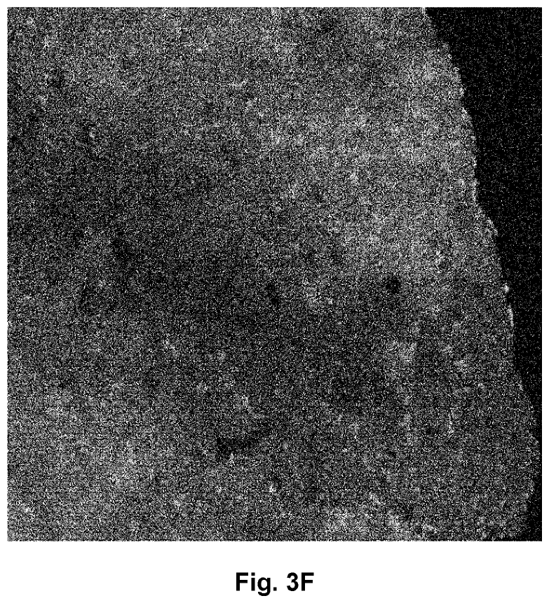

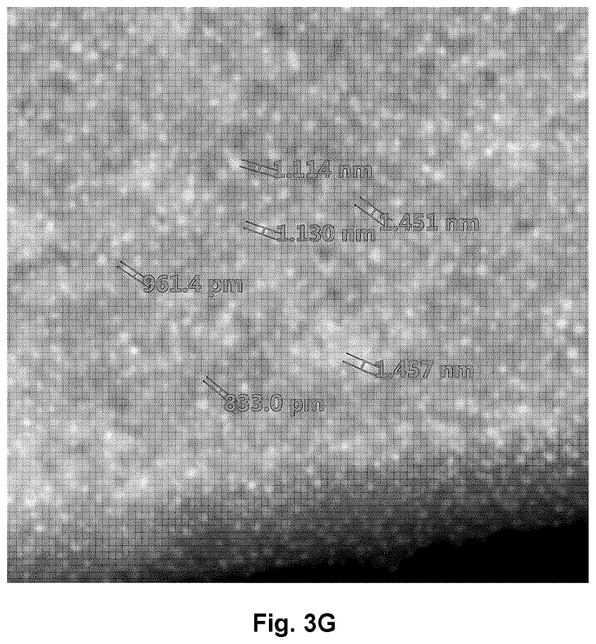

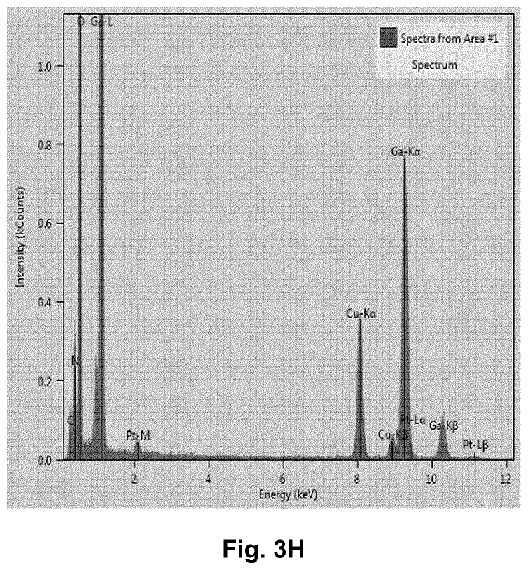

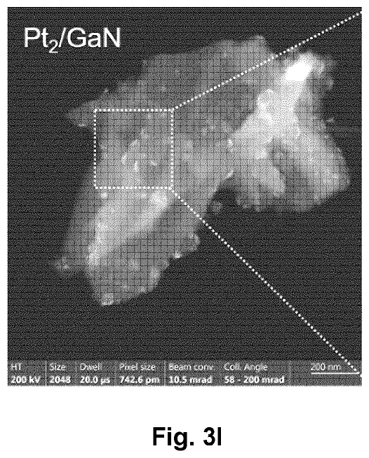

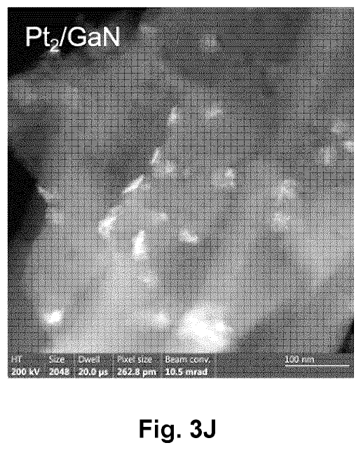

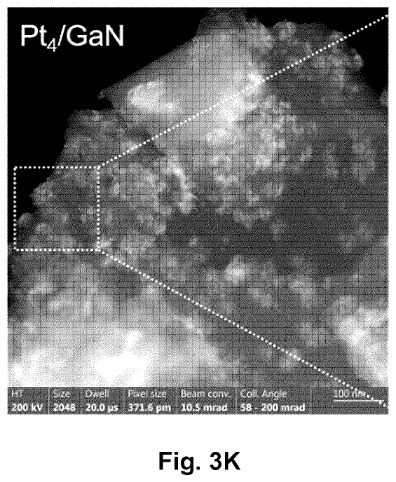

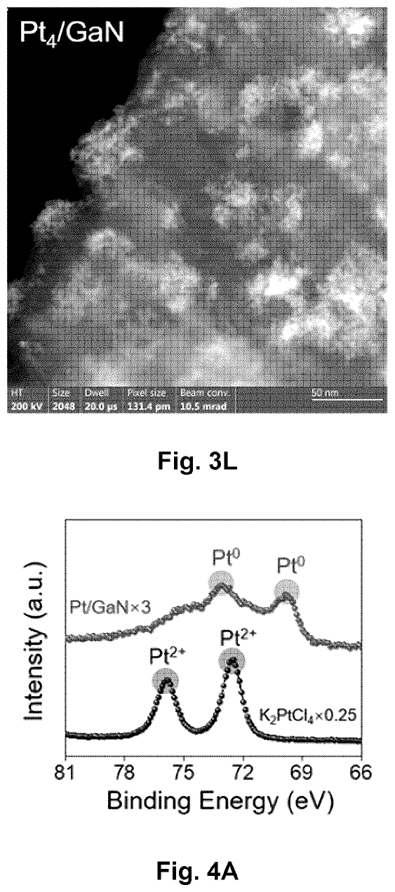

The morphologies of the exemplary 1 wt % Pt/GaN catalyst are shown in FIG. 3A. The microscopic characterization indicated that Pt was deposited on the surface of GaN as cluster with the average particle size of about 0.9-1.4 nm (FIG. 3G). The small size of the Pt particle revealed a larger surface area, which is beneficial for enhancing the activity of the catalyst. The HAADF-STEM image and the EDXS mapping showed that Pt clusters were well dispersed on the surface of GaN nanoparticles (FIGS. 3B, 3C, 3D, 3E, and 3F). The EDXS spectrum corroborated the microscopy findings confirming the deposition of Pt metal on the surface of GaN (FIG. 3H). It was also observed by HAADF-STEM that high metal loading samples shown obvious metal aggregation to form metal “island” (FIGS. 3I-3L).

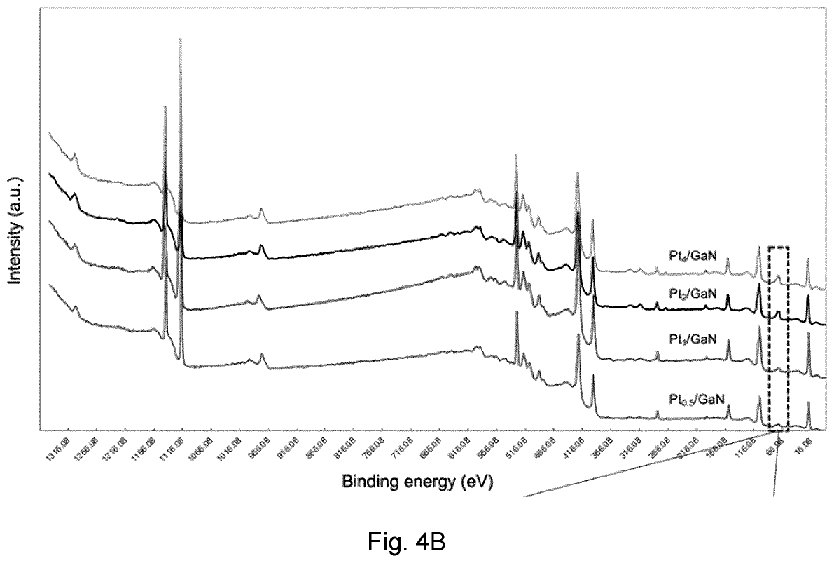

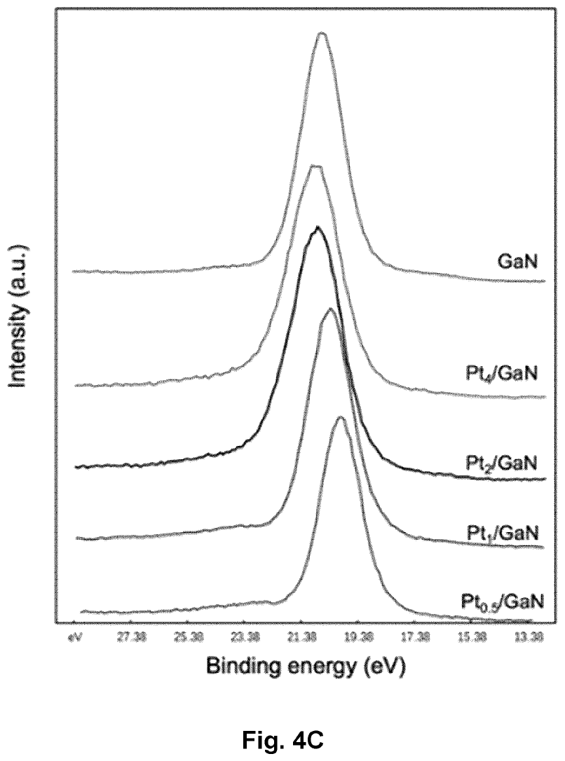

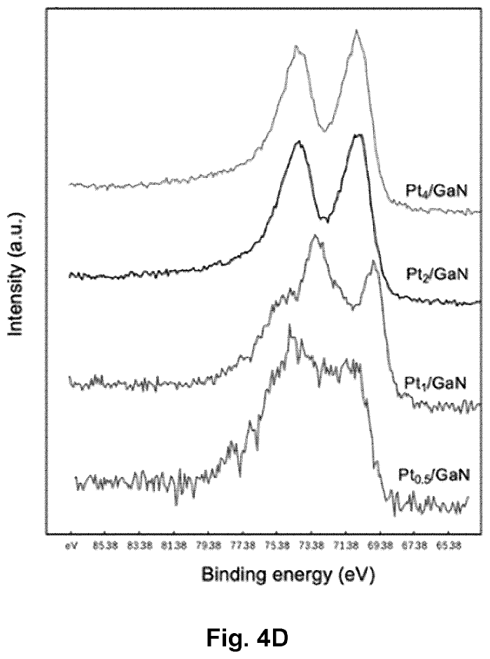

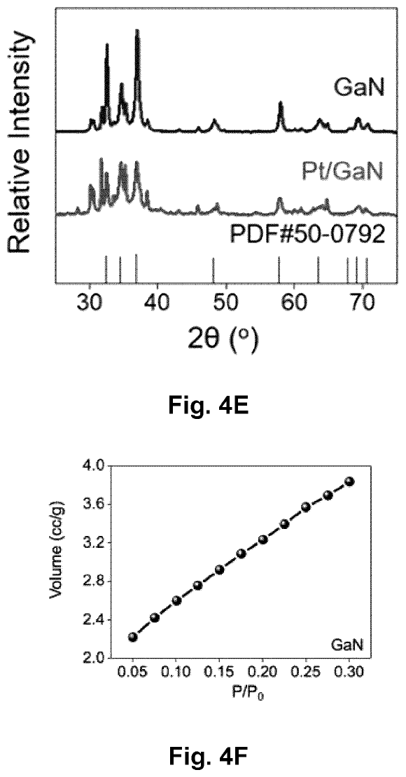

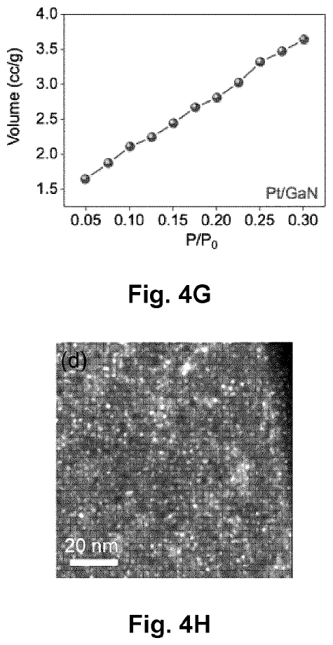

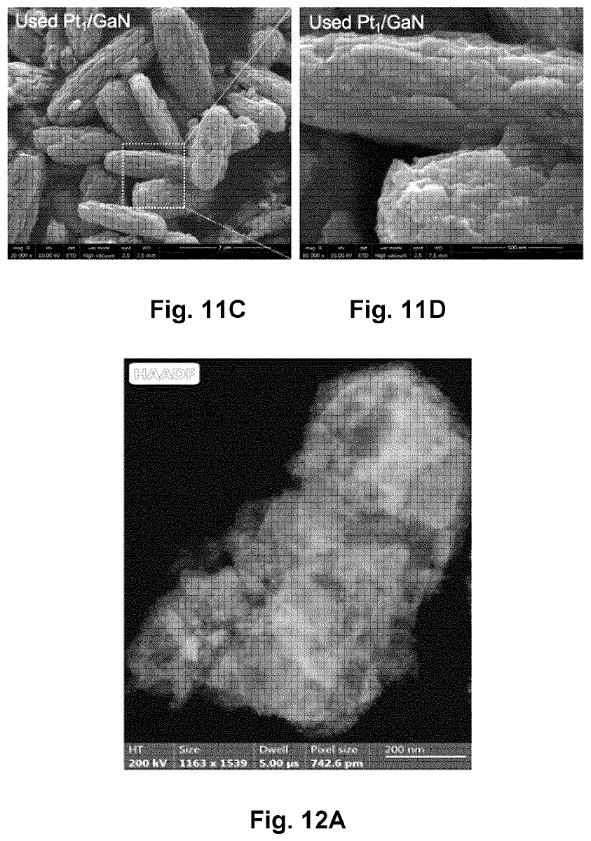

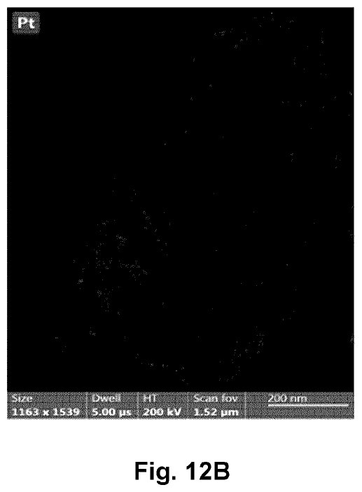

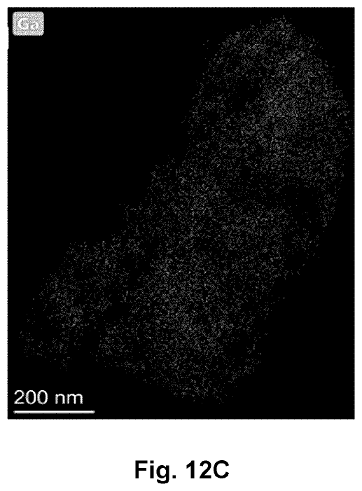

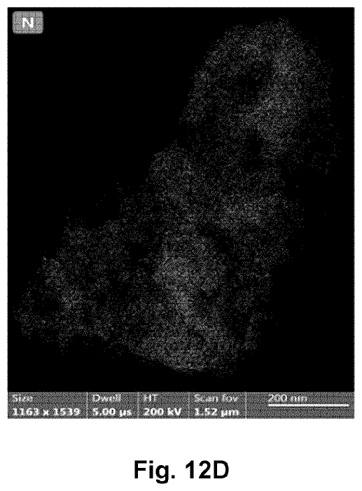

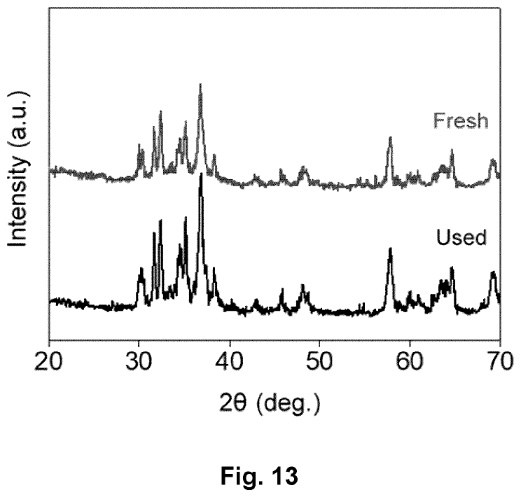

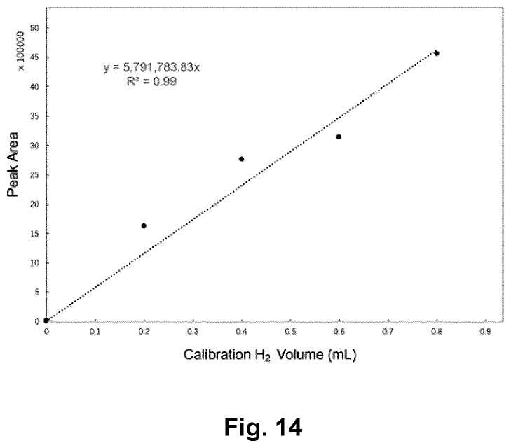

The X-ray photoelectron spectroscopy (XPS) analysis indicated that Pt metal with zero-oxidation state was successfully introduced with assistance of GaN-semiconductor as photosensitizer (FIGS. 4A-4D). As shown in the Powder X-ray diffraction (XRD) results (FIG. 4E), the GaN powder with typical wurtzite structure was well maintained even after metal loading, demonstrating high tolerance and stability of GaN support against photochemical reaction media. At the same time, XRD pattern of Pt/GaN sample showed no obvious characteristic peak for Pt nanoparticles which revealed the presence of metal Pt in the form of nanoclusters. The Brunauer-Emmett-Teller (BET) specific surface of Pt/GaN came out to be 12.31 m²/g whose value is similar with that of GaN (12.28 m²/g) (FIGS. 4F-4G), accordingly excluding the Pt nanoparticles and aggregation in the samples. This deduction was further reflected by the high-angle annular dark-field scanning transition electron microscopy (HAADF-STEM) image of Pt/GaN in which ultra-small Pt nanoclusters with the average particle size of ˜1.46 nm were identified on the GaN surface (FIGS. 4H-41). The energy-dispersive X-ray spectroscopy (EDXS) mapping files showed that Pt nanoclusters homogeneously dispersed on the entire GaN support (FIGS. 3E-3F), which was consistent with the HAADF-STEM results. These results demonstrated that the representative Pt/GaN sample was composed of ultra-small Pt clusters and typical wurtzite structured GaN. The amount of Pt metal precursors was controlled in the photoreduction reaction for optimizing the effective Pt/GaN boundary exposure to gas reactant. As-generated Ptₓ/GaN samples with different metal weight percentages (x=0.5%, 1%, 2% and 4%) relative to GaN were used to catalyze the methane transformation.

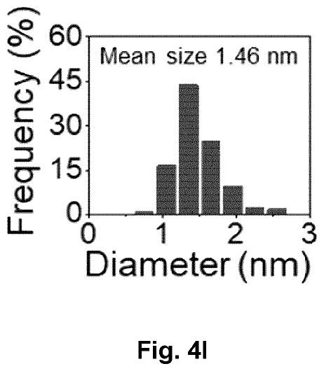

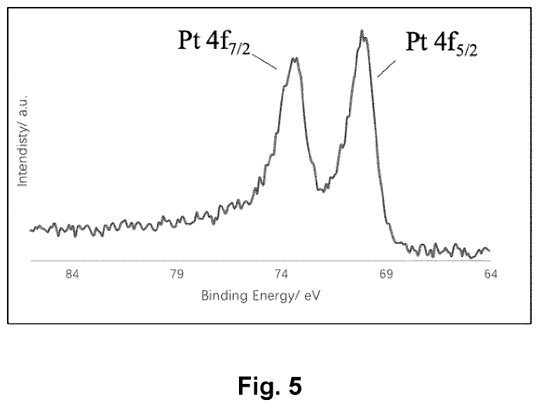

The electronic properties of the 1 wt % Pt/GaN catalyst were investigated by XPS. Then XPS results showed that the Pt 4f peaks are negatively shifted (73.3 eV and 70.2 eV; FIG. 5) compared to the standard binding energies of platinum (74.8 eV and 71.2 eV). This indicated that Pt clusters on the GaN surface possesses a higher electron density, which are beneficial to the activation of H₂ gas for the hydrogenation of benzene. The enhanced hydrogenation activity of Pt was tested by injecting 0.1 mmol of benzene and 2 mmol of hydrogen gas into the 50 mL reactor at 300° C. After 2 hours, about 90% of benzene was converted to cyclohexane.

### Example 3: Catalytic Activity Analysis

Schlenk glassware or vacuum line techniques were used to assess the catalytic activity of the catalysts. Methane in this experiment was dried by passing through a column of MgSO₄ and CuSO₄ before the catalyst activity test. The catalyst activity test was performed in a 50 mL round bottom reactor at 200° C., 250° C., 300° C., 350° C., 400° C., 500° C. and 600° C. First, 20 mg of the catalysts were dispersed evenly at the bottom of the reactor and the closed reactor was heated to 400° C. under vacuum for 2 hours to remove the remaining water and other gas molecules on the materials. The reactor was cooled to room temperature under vacuum. The catalysts were purged with methane and were vacuumed three times before 2 mmol of methane was injected. During the reaction, the reactor was heated to the desired temperature and kept for 2 hours. The organic products were in-situ analyzed and quantified by gas chromatography-mass spectrometry (GCMS) with flame ionization detector (FID). For the reusability test, the used catalysts were recycled at 500° C. under vacuum for 3 hours after each cycle to remove all the remaining reactant and products. Subsequently, the reactor was cooled to room temperature under vacuum. The catalysts were purged with methane and were vacuumed three times before 2 mmol of methane was injected. The selectivity of cyclohexane was determined as the ratio of moles of cyclohexane produced in the reaction to the total mole of all hydrocarbon products in terms of carbon (as shown below).

$$Selectivity of cyclohexane = \frac{Moles of cyclohexane produced in terms of carbon}{Moles of all hydrocarbon products in terms of carbon} \times 100 \% .$$

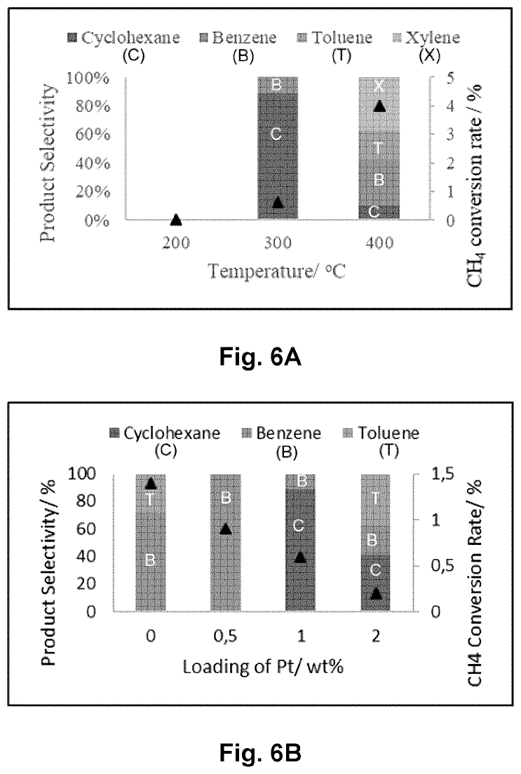

The catalytic activity of 1 wt % Pt/GaN for the conversion of methane to cyclohexane was investigated in a closed reactor at 200° C., 300° C. and 400° C. for 2 hours. As shown in FIG. 6A, at 400° C., the major organic products detected were mainly aromatics (benzene (17.1%), toluene (23.3%), xylene (41.7%)), cyclohexane (11.9%), and a mixture of short-chain hydrocarbons less than C6 (6.0%). Under the high temperature (>300° C.), the dehydrogenation of alkane was more favorable and enhanced the desorption of hydrogen and benzene on the surface of the solid materials, which induced the lower selectivity towards cyclohexane. At 300° C., much to the inventors' surprise, a high selectivity of cyclohexane was achieved (88.9%), with the byproduct as benzene (8.1%) and a mixture of short-chain hydrocarbons less than C6 (3.0%). The carbon oxides side products, which generally originate from impurities in the initial raw GaN, were negligible during the reaction. When the reaction was carried out at a temperature of 200° C. or lower, the activity of the catalyst was low (below 1%).

Next, various amount of Pt metal was deposited on GaN nanoparticles to investigate the optimal loading of co-catalyst. When the loading of Pt was reduced to 0.5 wt %, the catalyst could not catalyze the reaction. As shown in FIG. 6B, when the loading of Pt was increased to 2 wt %, the major products were cyclohexane (59.5%), benzene (12.3%), toluene (21.9%), and a mixture of short-chain hydrocarbons less than C6 (6.3%). The methane conversion rate decreased from 0.9% to 0.2% when the loading of Pt increased from 0.5 wt % to 2 wt %. Without Pt, no cyclohexane was formed during the reaction and benzene (71.0%) and toluene (29%) were the final product with methane conversion rate of 1.4%.

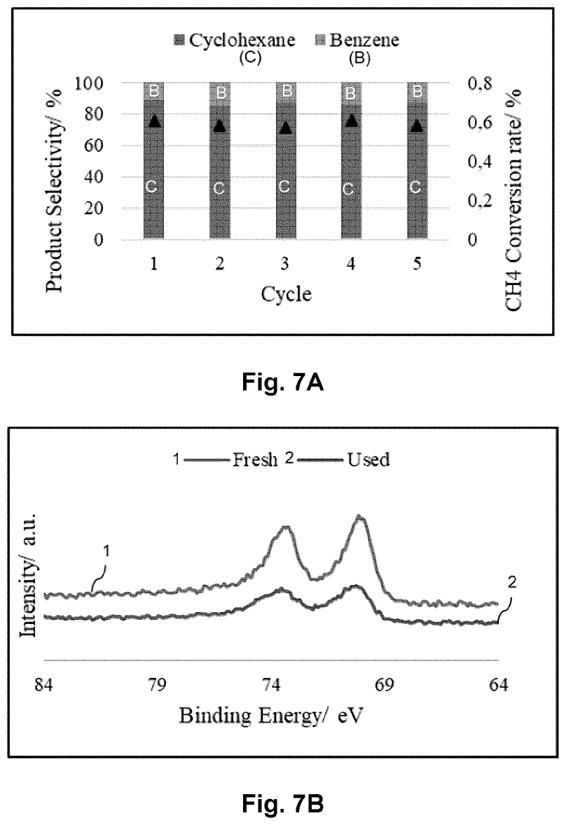

The reusability of the as-prepared 1 wt % Pt/GaN was investigated (FIGS. 7A-7B). After 5 runs of reaction, no significant change of selectivity or conversion rate was observed (FIG. 7A). Moreover, the XPS spectra of Pt 4f for the 1 wt % Pt/GaN remained the same (FIG. 7B). This result demonstrated that the electronic properties of Pt clusters were consistent after the long reaction time.

In summary, the direct transformation from methane to cyclohexane with high selectivity was achieved. A 88% selectivity towards cyclohexane was obtained with the 1 wt % Pt/GaN catalyst under 300° C. with excellent reusability.

### Example 4 Catalyst Performance and Comparison

Comparative example catalysts were prepared with GaN and metals other than Pt. A photodeposition method was also applied to deposit Pd, Cu, Ag, and Au on GaN nanoparticles on separate substrates for comparison. During the preparation, the corresponding metal precursors (H₂PdCl₄, CuCl₂, AgNO₃, H₂AuCl₄·3H₂O) with a loading of 1 wt. % was dissolved in 6 mL of deionized water and 2 mL of methanol as stock solution. Then, 20 mg of the GaN nanoparticles was dispersed in 4 ml of the stock solution in a quartz tube and was stirred under photoirradiation for 3 hours under a xenon lamp (PE300 BUV) in argon. The suspension was collected by centrifuge and was washed with distilled water and methanol several times and was dried under vacuum at 100° C. overnight.

The loading of Ru co-catalyst was conducted via the impregnation method. In the typical synthesis of 1 wt % Ru/GaN, 0.63 mg of Ru₃(CO)₁₂ was dissolved in dry tetrahydrofuran (THF) (2 mL). Then, 20 mg of the GaN nanoparticles was dispersed in the solution and stirred for 2 hours. In order to completely remove the carbonyl groups and other impurities, the reaction mixture was then heated to 200° C. under vacuum and held for 1 hour. Afterwards, the temperature was raised to 350° C., held for 2 hours and then cooled to room temperature.

The loading of Pt, Pd, and Ag on other catalysts supports (Ga₂O₃, TiO₂, ZnO, Al₂O₃, zeolite) were performed via a chemical reduction method. During the preparation, the corresponding metal precursors (H₂PtCl₄, H₂PdCl₄, AgNO₃) with the desired loading were dissolved in 50 mL of aqueous solution of 0.20 M NaOH and 0.20 M NaBH₄ as stock solution. Then, 20 mg of the solid materials was dispersed in 5 mL of the stock solution and was vigorously stirred at room temperature. After one hour of stirring, the solid materials were collected by centrifugation and were washed with distilled water and methanol several times and then dried under vacuum at 100° C. overnight.

Besides Pt metal, other metals with a hydrogenation capacity were also deposited on GaN. It was found that there was no production of cyclohexane or aromatics detected for 1 wt % of Cu, Ag, Pd, Ru, and Au metal deposition after 2 hours' reaction time. In addition, 1 wt % of Pt/C was mechanically mixed with GaN and the major organic products were benzene (72.4%) and toluene (27.6%), which revealed the importance of the interaction between Pt clusters and GaN.

Ga₂O₃, TiO₂, ZnO, Al₂O₃, and zeolite are commonly applied as catalyst support for a heterogeneous catalyst. In this experiment, 1 wt % loading of Pt, Pd and Ag metals were deposited on those solid materials and their performance was compared towards the synthesis of cyclohexane from methane with the as-prepared 1 wt % Pt/GaN. Surprisingly, 1 wt % Pt/Ga₂O₃ and 1 wt % Pt/ZnO produced respectively 49% and 44% of cyclohexane (the major component) at 300° C., but the other catalysts only produced short-chain hydrocarbons (<C₆) and aromatic hydrocarbons as the major products. Pt cluster concentrations of 0.75, 1.25, 1.5, 2, and 4 wt. % were tested and found to be adequate, i.e. producing cyclohexane as a major component. Although the temperature of 300° C. was determined is an optimal temperature, temperatures of 250° C. and 350° C. were also found to be suitable.

The results are summarized in the table below:

TABLE 1
Summary of results

| Catalyst (20 mg) | Temperature | Main products after 2 h |
|---|---|---|
| 2 wt % Pt/GaN | 300° C. | benzene (12%), cyclohexane |
| | | (60%), toluene (22%), |
| | | mixture of short-chain |
| | | hydrocarbons less than C6 (6%) |
| 2 wt % Pt/GaN | 400° C. | Benzene (14%), cyclohexane |
| | | (3%), toluene (17%), |
| | | xylene (64%), mixture of |
| | | short-chain hydrocarbons less |
| | | than C6 (2%) |
| 2 wt % Pt/GaN | 200° C. | N/A |
| 1 wt % Pt/GaN | 300° C. | Benzene (8%), cyclohexane |
| | | (89%), mixture of short-chain |
| | | hydrocarbons less than C6 (3%) |
| 1 wt % Pt/GaN | 200° C. | N/A |
| 1 wt % Pt/GaN | 250° C. | Benzene (24%), cyclohexane |
| | | (63%), mixture of short-chain |
| | | hydrocarbons less than C6 (13%) |
| 1 wt % Pt/GaN | 350° C. | Benzene (42%), cyclohexane |
| | | (38%), toluene (13%), |
| | | mixture of short-chain |
| | | hydrocarbons less than C6 (7%) |
| 1 wt % Pt/GaN | 400° C. | Benzene (17%), cyclohexane |
| | | (12%), toluene (23%), |
| | | xylene (42%), mixture of |
| | | short-chain hydrocarbons less |
| | | than C6 (6%) |
| 1 wt % Pt/GaN | 100° C. | N/A |
| 1 wt % Pt/GaN | 500° C. | Benzene (76%), cyclohexane |
| | | (4%), toluene (9%), xylene |
| | | (1%), mixture of short-chain |
| | | hydrocarbons less than |
| | | C6 (10%) |
| 1 wt % Pt/GaN | 600° C. | Benzene (82%), toluene (12%), |
| | | xylene (1%), mixture of |
| | | short-chain hydrocarbons |
| | | less than C6 (5%) |
| 4 wt % Pt/GaN | 300° C. | Benzene (44%), cyclohexane |
| | | (49%), mixture of short-chain |
| | | hydrocarbons less than C6 (7%) |
| 5 wt % Pt/GaN | 300° C. | Benzene (26%), cyclohexane |
| | | (6%), mixture of short-chain |
| | | hydrocarbons less than C6 (68%) |
| 6 wt % Pt/GaN | 300° C. | Benzene (3%), mixture of |
| | | short-chain hydrocarbons |
| | | less than C6 (97%) |
| 0.5 wt % Pt/GaN | 300° C. | Benzene (93%), mixture of |
| | | short-chain hydrocarbons |
| | | less than C6 (7%) |
| 0.75 wt % Pt/GaN | 300° C. | Benzene (18%), cyclohexane |
| | | (74%), mixture of short-chain |
| | | hydrocarbons less than C6 (8%) |
| 1.25 wt % Pt/GaN | 300° C. | Benzene (28%), cyclohexane |
| | | (66%), mixture of short-chain |
| | | hydrocarbons less than C6 (6%) |
| 1.5 wt % Pt/GaN | 300° C. | Benzene (20%), cyclohexane |
| | | (54%), toluene (21%), |
| | | mixture of short-chain |
| | | hydrocarbons less than C6 (5%) |
| 0.5 wt % Pt/GaN | 400° C. | Benzene (24%), cyclohexane |
| | | (6%), toluene (27%), |
| | | xylene (35%), mixture of |
| | | short-chain hydrocarbons less |
| | | than C6 (8%) |
| 0.5 wt % Pt/GaN | 200° C. | N/A |
| 1 wt % Cu/GaN | 300° C. | N/A |
| 1 wt % Ag/GaN | 300° C. | N/A |
| 1 wt % Pd/GaN | 300° C. | C9 146m/z |
| 1 wt % Ru/GaN | 300° C. | N/A |
| 1 wt % Au/GaN | 300° C. | N/A |
| GaN + 5% Pt/C | 300° C. | Benzene (66%), toluene (25%), |
| | | mixture of short-chain |
| | | hydrocarbons less than C6 (9%) |
| ZnO | 300° C. | Benzene (62%) toluene (14%), |
| | | mixture of short-chain |
| | | hydrocarbons less than C6 (24%) |
| Ga₂O₃ | 300° C. | Benzene (34%), mixture of |
| | | short-chain hydrocarbons |
| | | less than C6 (66%) |
| TiO₂ | 300° C. | N/A |
| 1 wt % Pt/ZnO | 300° C. | Benzene (51%), cyclohexane |
| | | (44%), mixture of short-chain |
| | | hydrocarbons less than C6 (5%) |
| 1 wt % Pt/TiO₂ | 300° C. | N/A |
| 1 wt % Pt/Ga₂O₃ | 300° C. | Benzene (44%), cyclohexane |
| | | (49%), mixture of short-chain |
| | | hydrocarbons less than C6 (7%) |
| 1 wt % Pt/Zeolite | 300° C. | N/A |
| 1 wt % Pt/Graphite | 300° C. | N/A |
| 1 wt % Pt/C₃N₄ | 300° C. | Benzene (80%), mixture of |
| | | short-chain hydrocarbons |
| | | less than C6 (20%) |
| 1 wt % Pt/Al₂O₃ | 300° C. | Benzene (8%) toluene |
| | | (22%) xylene (70%) |

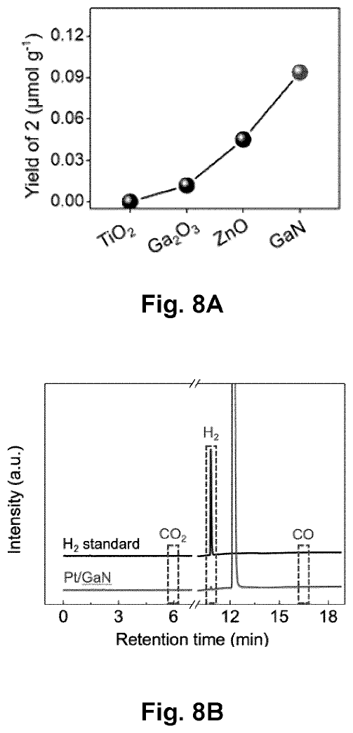

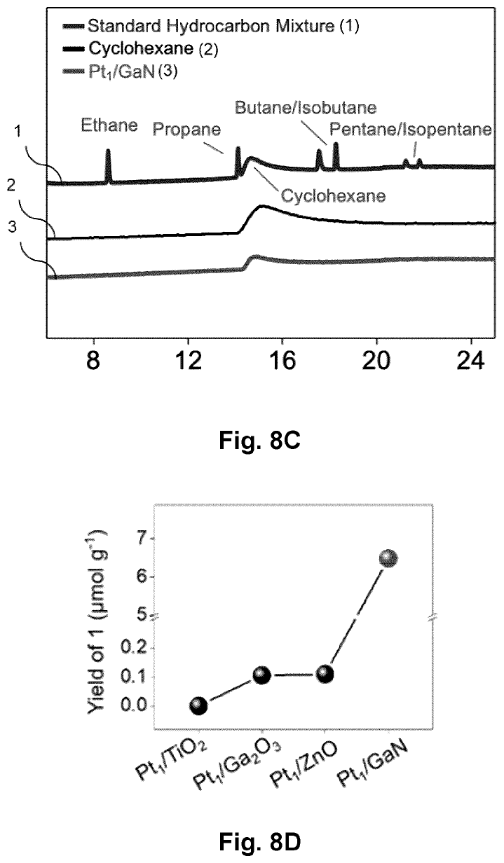

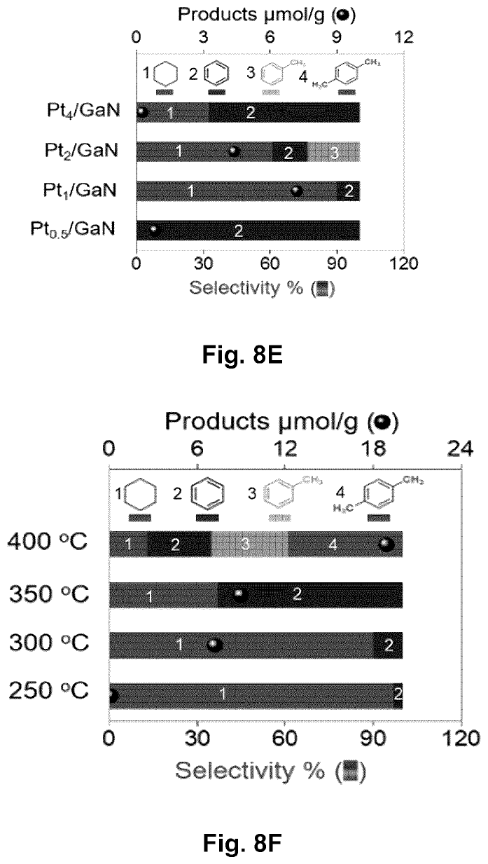

Additional testing was performed by performing a thermal-catalytic methane non-oxidative conversion with different semiconductor-supports in a closed reactor at 300° C. for 2 h, in which 20 mg of Pt/GaN was homogeneously coated on the reactor wall using 2 mmol of high-purity methane as the single reactant. As shown in FIG. 8A (Entries 1-4 of Table 2), GaN support exhibited a higher yield of benzene compared to other supports for thermal methane conversion. In the final products analysis, only aromatics including benzene (blue) toluene (orange) and xylene (green) as side-products, and hydrogen gas were generated (FIG. 8B). As shown in FIG. 8B the main by-product is H₂ with no generation of oxidation and/or over-oxidation products like carbon oxide and/or carbon dioxide, demonstrating GaN has strong ability for the direct activation of methane and can push methane aromatization conversion to generate benzene and H₂ gas. Negligible carbon oxides and hydrocarbon gas were produced but hydrogen gas was observed after the reaction (FIGS. 8B and 8C). These results demonstrated that GaN support indeed plays an essential role for activating inert methane molecules to form aromatic compounds that will be beneficial for the further cyclohexane and hydrogen gas generation.

TABLE 2
Results of various catalysts for the methane to cyclohexane conversion

| | | | Selectivity of | Yield (μmol/g) | | | |
|---|---|---|---|---|---|---|---|
| | | Temperature | cyclohexane | Cyclo- | | | |
| Entry | Catalyst | (° C.) | (%) | hexane | Benzene | Toluene | Xylene |
| 1 | GaN | 300 | / | n.d. | 0.094 | traces | traces |
| 2 | TiO₂ | 300 | / | n.d. | n.d. | n.d. | n.d. |
| 3 | Ga₂O₃ | 300 | / | n.d. | 0.012 | n.d. | n.d. |
| 4 | ZnO | 300 | / | n.d. | 0.045 | 0.0085 | n.d. |
| 5 | Pt₁/TiO₂ | 300 | / | n.d. | n.d. | n.d. | n.d. |
| 6 | Pt₁/Ga₂O₃ | 300 | 47.83 | 0.11 | 0.12 | n.d. | n.d. |
| 7 | Pt₁/ZnO | 300 | 42.31 | 0.11 | 0.15 | n.d. | n.d. |
| 8 | Pt₁/GaN | 300 | 90.14 | 6.49 | 0.71 | n.d. | n.d. |
| 9 | Pt_{0.5}/GaN | 300 | / | n.d. | 0.83 | n.d. | n.d. |
| 10 | Pt₂/GaN | 300 | 61.42 | 2.69 | 0.68 | 1.01 | n.d. |
| 11 | Pt₄/GaN | 300 | 42.31 | 0.11 | 0.15 | n.d. | n.d. |
| 12 | Pt₀/GaN | 300 | / | n.d. | 0.21 | n.d. | n.d. |
| 13 | Pt₁/GaN | 250 | 97.22 | 0.084 | 0.0024 | n.d. | n.d. |
| 14 | Pt₁/GaN | 350 | 37.05 | 3.32 | 5.64 | n.d. | n.d. |
| 15 | Pt₁/GaN | 400 | 12.72 | 2.39 | 4.22 | 4.85 | 7.44 |
| 16 | Pt₁/GaN | 300 | 93.52 | 11.55 | 0.80 | n.d. | n.d. |
| 17 | Pt₁/GaN | 300 | 92.23 | 41.42 | 3.49 | n.d. | n.d. |
| 18 | Pt_{0.75}/GaN | 300 | 61.80 | 0.85 | 1.13 | n.d. | n.d. |

n.d. = not detectable
Pt₁ = 1 wt. % Pt
Pt_{0.5} = 0.5 wt. % Pt
Pt_{0.75} = 0.75 wt. % Pt
Pt₄ = 4 wt. % Pt

Introducing Pt metal on to the methane-active semiconductor-support to form Pt/GaN interface prompted the challenging generation of cyclohexane from methane with a high selectivity. No desired cyclohexane was formed at 300° C. by the methane-nonactive Pt/TiO₂ sample (FIG. 8D and Entry 5 of Table 2). In contrast, Pt-modified methane-active supports showed noticeable production of cyclohexane under the same reaction conditions (FIG. 8D and Entries 6-8, and 18 of Table 2), in which Pt/GaN hybrids exhibits 25-fold higher catalytic performance of 6.49 μmol g⁻¹ for methane conversion than those of well-controlled Pt/support (here are Ga₂O₃ and ZnO) heterojunctions. All the results showed that GaN can serve as the preferred methane-active support to load Pt nanoclusters used for the hydrogenation of methane-aromatized benzene to cyclohexane.

The further optimization of metal loading on GaN surface was conducted to finely accommodate both electron density of Pt and electrostatic polarity of Ga—N pair for well matching yield rate of methane dehydrogenation and benzene hydrogenation, thus ensuring Pt/GaN with high chemoselectivity and productivity for methane-to-cyclohexane conversion (FIG. 8E). The low metal content of Pt_{0.5}/GaN sample mainly with appreciable electron-deficiency Pt cluster (FIG. 4D) and enhanced methane-active Ga—N pairs (FIG. 4C) could not generate cyclohexane due to the poor hydrogenation capacity of positively charged Pt nanoclusters (Entries 9 of Table 2). In combination with trace yield of PtO/GaN for cyclohexane synthesis, the positive contribution of Pt²⁺ species in Pt clusters for methane-to-cyclohexane transformation can be eliminated. The Pt concentration varying from 0.5 to 1 wt % was able to result in the generation of highly efficient Pt—GaN interface between electron-rich Pt clusters and strong Ga—N pair modified GaN support. That has been confirmed by the downward shift of Pt XPS peaks and slight upshift of Ga XPS peaks from Pt_{0.5}/GaN to Pt₁/GaN. (FIGS. 4B-4D) Such “two-in-one” interface of Pt₁/GaN coupled with hydrogenation-active Pt cluster and methane-active GaN therefore achieved the best-performance in both productivity (6.49 μmol/g) and selectivity (90%) toward cyclohexane as well as a considerable hydrogen gas production (5.5 μmol/g). Such an excellent reactivity far outperforms all reported catalytic systems even handled above 600° C. (Table 3). Further increasing the Pt loading on the surface of GaN inevitably induced aggregation of metal clusters that can further impede photogenerated electron transfer from GaN to Pt (FIGS. 4B-4D), thus losing their catalytic reactivity at the electron-deficiency Pt—GaN boundary (FIG. 8E and Entries 10-11 of Table 2). These results demonstrate the synergetic role of highly dispersed Pt₁/GaN interface with strong Ga—N pair modified GaN support and electron-rich Pt clusters on simultaneously catalyzing the methane aromatization and the subsequent hydrogenation for synthesizing cyclohexane in an ultra-selective manner.

TABLE 3
Comparison of catalytic performance of different catalysts
for direct methane to cyclohexane conversion

| | | Selectivity to | Cyclohexane | |
|---|---|---|---|---|
| | Reaction | cyclohexane | productivity | |
| Catalysts | Condition | (%) | (μmol/g) | Ref. |
| Pt₁/GaN | 300° C., 1 atm | 90.14 | 6.49 | N/A |
| GaN² | 650-710° C. | Detectable | / | Catal. |
| | flow (GHVS | | | Commun. |
| | of 114-720 | | | 2018, 106, |
| | ml_{N} g_{cat}⁻¹h⁻¹), | | | 16-19. |
| | 1 atm | | | |
| GaN³ | 700° C., 1 atm | 1.1 | 0.275 | Angew. |
| | | | | Chem., |
| | | | | 2014, |
| | | | | 126, |
| | | | | 14330- |
| | | | | 14333. |

The reaction temperature has an impact on the catalytic balance i.e. methane aromatization and benzene hydrogenation rates (FIG. 8F and Entries 13-15 of Table 2). Cyclohexane selectivity (97%) was achieved at a lower temperature of 250° C. but at a small cyclohexane productivity was observed (Entry 13 of Table 2). Further increasing reaction temperature for breaking thermodynamic limitation at low temperature benefits to enhance methane conversion applied in practical manufacture. When the reaction temperature was raised to 300° C. (Entry 8 of Table 2), the cyclohexane generation rate reached an optimal value (6.49 μmol g⁻¹) in the temperature-dependent reactivity profiles wherein superior chemoselectivity was comparable to the one at 250° C. However, higher temperatures of above 300° C. to 350° C. even 400° C. were found to lower selectivity (42% and 12%) as well as the corresponding yield (3.32 μmol g⁻¹ and 2.39 μmol g⁻¹) toward cyclohexane in our catalytic case (Entries 14-15 of Table 2). Consequently, the subsequent experiments were performed at 300° C. Such reaction temperature-dependent trade-off correlation between cyclohexane selectivity and productivity presented by the optimized Pt/GaN again imply the presence of a synergistic mechanism working at the Pt/GaN interface which is responsible for the improvement observed.

Pt₁/GaN catalyst was still capable of maintaining good selectivity (93% and 92%) and displaying higher productivity (11 μmol g⁻¹ and 41 μmol g⁻¹) after updating batch reactor from 50 mL (Entries 1-15 of Table 2) to 100 mL (Entry 16 of Table 2) and even 500 mL (Entry 17 of Table 2). Accordingly, the utilization of Pt₁/GaN catalyst in combination with an effective reactor enables the scale-up production of cyclohexane in an appreciable yield.

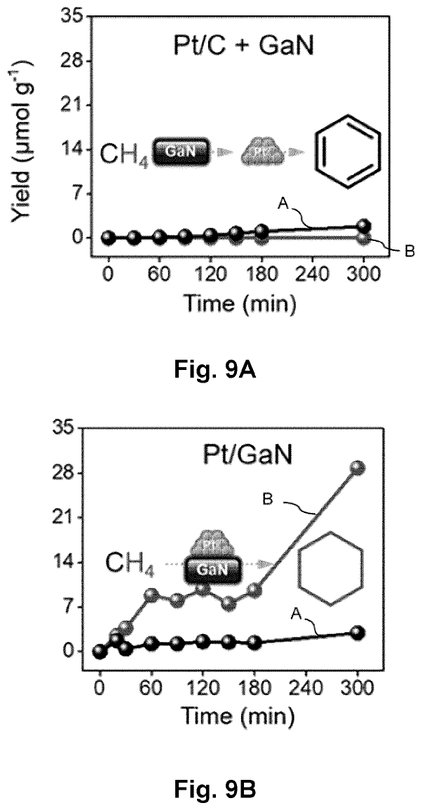

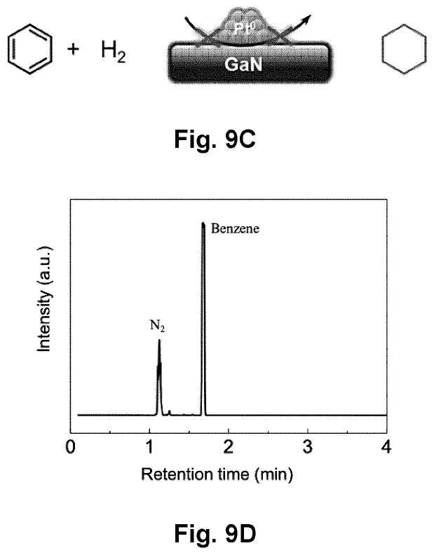

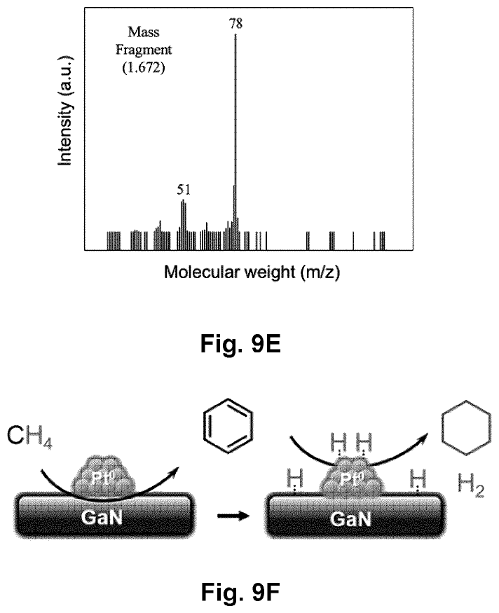

To gain in-depth insight of the catalytic methane transformation process on Pt/GaN surface, the time-dependent reaction performance for as-formed Pt/GaN was compared to a mechanical mixture of commercial Pt/C and bare GaN (FIGS. 9A and 9B and Tables 4-5) of which there is similar Pt loading. It was found that only trace non-oxidative methane conversions occurred in the presence of Pt/C and GaN mixture even after 5 h under the standard thermal conditions owing to the non-existence of the effectively integrated interface between Pt/C and GaN (FIG. 9A). In contrast, the yield of cyclohexane was increased when using Pt/GaN and was accompanied with a small amount of benzene generated from aromatization of methane (FIG. 9B). Such results demonstrated that the overall reaction pathway indeed involves successive aromatization and hydrogen transfer steps near the boundary of Pt clusters and GaN support. A further control experiment was carried out to explore the surface hydrogen auto-transfer process. Equivalent amounts of benzene and H₂ gas were found to match those of theoretical production of the methane liquefaction reaction over Pt/GaN (FIG. 9C). The hydrogenation of benzene towards cyclohexane with hydrogen gas did not take place (FIGS. 9D-9E). These results unravel that the in-situ generated H-atoms on the surface of GaN via methane dehydroaromatization are quite prone to migrate to the adjacent Pt metal surface for the subsequent hydrogenation driven by active metal hydride intermediate rather than undergoing two separate steps of dehydrogenation-hydrogenation to accomplish the successive hydrogenation of benzene (FIG. 9F).

TABLE 4
Results of methane transformation into cyclohexane over Pt₁/GaN

| | | | Selectivity of | Yield (μmol/g) | | | |
|---|---|---|---|---|---|---|---|
| | | Temperature | cyclohexane | Cyclo- | | | |
| Entry | Catalyst | (° C.) | (%) | hexane | Benzene | Toluene | Xylene |
| 1 | Pt₁/GaN | 10 | / | n.d. | 1.43 | n.d. | n.d. |
| 2 | Pt₁/GaN | 20 | 59.62 | 2.54 | 1.72 | n.d. | n.d. |
| 3 | Pt₁/GaN | 30 | 89.51 | 3.67 | 0.43 | n.d. | n.d. |
| 4 | Pt₁/GaN | 60 | 87.66 | 8.81 | 1.24 | n.d. | n.d. |
| 5 | Pt₁/GaN | 90 | 86.58 | 8.00 | 1.24 | n.d. | n.d. |
| 6 | Pt₁/GaN | 120 | 86.26 | 9.79 | 1.56 | n.d. | n.d. |
| 7 | Pt₁/GaN | 150 | 83.67 | 7.48 | 1.46 | n.d. | n.d. |
| 8 | Pt₁/GaN | 180 | 87.31 | 9.56 | 1.39 | n.d. | n.d. |
| 9 | Pt₁/GaN | 300 | 90.73 | 28.78 | 2.94 | n.d. | n.d. |

n.d. = not detectable

TABLE 5
Results of methane transformation into cyclohexane
over the mixture of commercial Pt/C and GaN

| | | | Selectivity of | Yield (μmol/g) | | | |
|---|---|---|---|---|---|---|---|
| | | Temperature | cyclohexane | Cyclo- | | | |
| Entry | Catalyst | (° C.) | (%) | hexane | Benzene | Toluene | Xylene |
| 1 | 5% Pt/C + GaN | 30 | / | n.d. | 0.0075 | n.d. | n.d. |
| 2 | 5% Pt/C + GaN | 60 | / | n.d. | 0.063 | n.d. | n.d. |
| 3 | 5% Pt/C + GaN | 90 | / | n.d. | 0.083 | n.d. | n.d. |
| 4 | 5% Pt/C + GaN | 120 | / | n.d. | 0.10 | n.d. | n.d. |
| 5 | 5% Pt/C + GaN | 150 | / | n.d. | 0.15 | n.d. | n.d. |
| 6 | 5% Pt/C + GaN | 180 | / | n.d. | 0.17 | n.d. | n.d. |
| 7 | 5% Pt/C + GaN | 300 | / | n.d. | 0.18 | n.d. | n.d. |

n.d. = not detectable

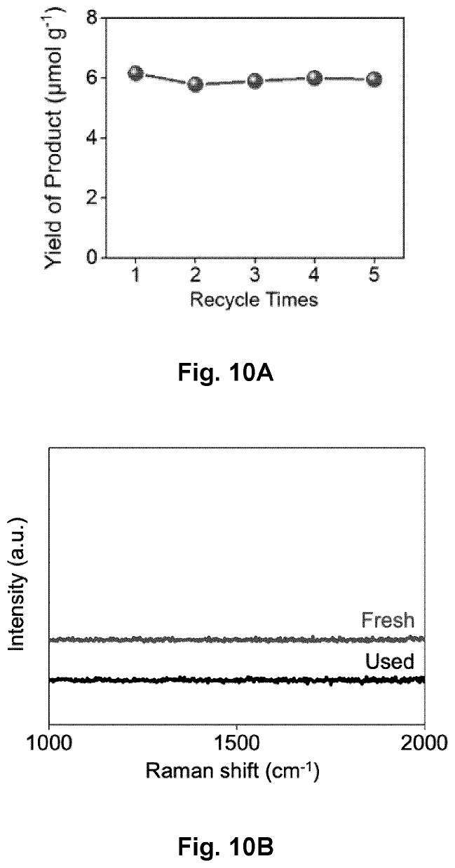

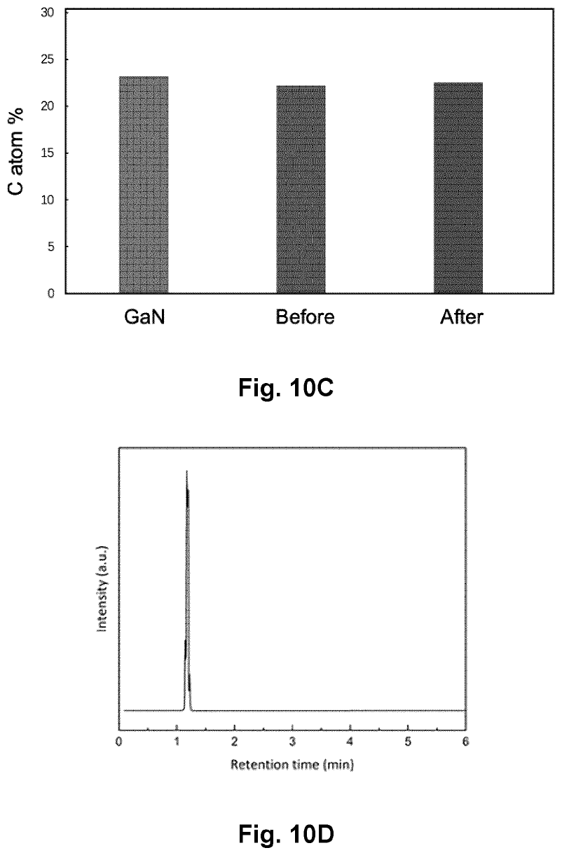

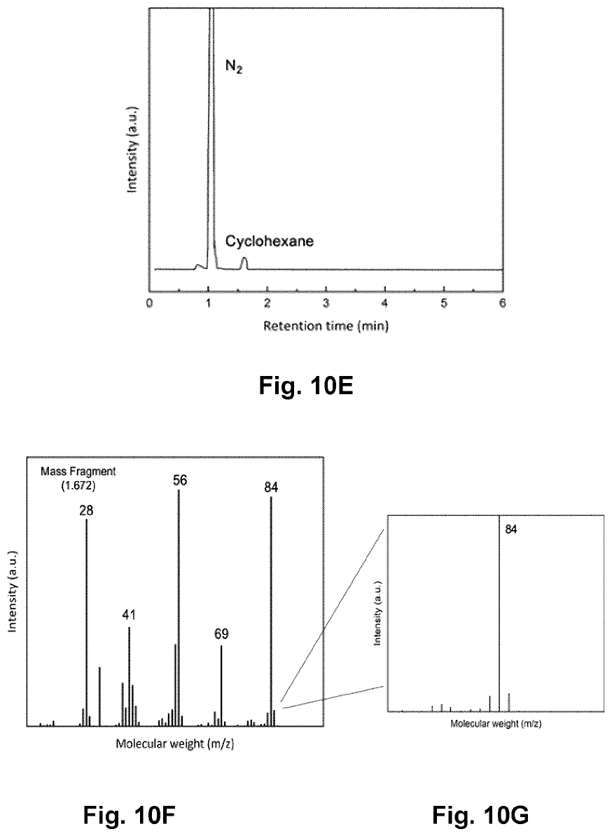

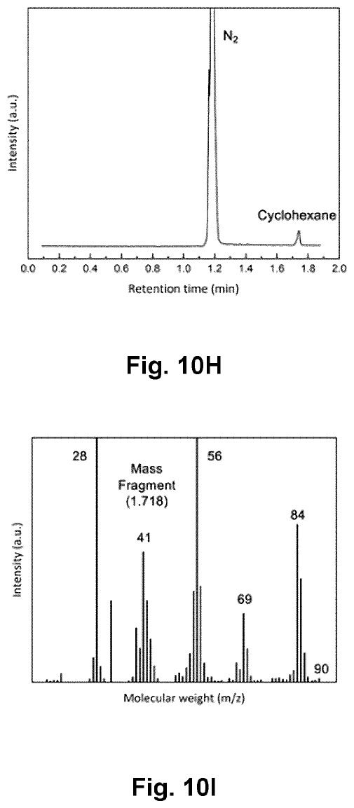

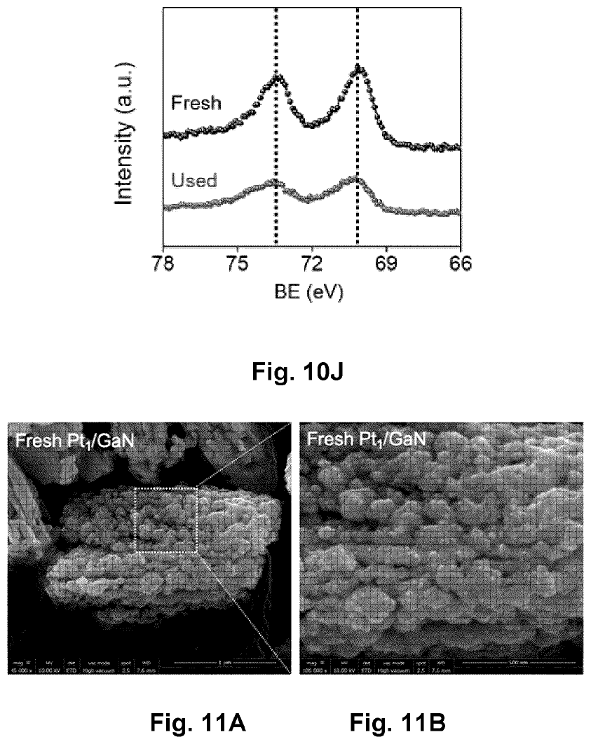

Given the nature of heterogeneous catalysts, the Pt/GaN interface showed good recyclability without attenuation in terms of production rate of cyclohexane under the thermal condition for up to 5 cycles (FIG. 10A and Table 6). In the present catalytic system, no carbon allotrope deposition on the surface of catalyst was identified (FIGS. 10B-10C) before and after the reaction. Performing control and isotopic experiments offers a direct evidence for the carbon resource of cyclohexane produced by methane conversion rather than other carbon contamination (FIGS. 10D-101). It is noted that no detectable cyclohexane product was found under a standard condition just by switching methane with argon. More importantly, the comparison results obtained in ¹²CH₄ and ¹³CH₄ isotopic labeling conversion (FIGS. 10E-10G) demonstrated the existence of ¹³C labeling cyclohexane from direct methane conversion catalyzed by Pt/GaN. Simultaneously, the morphology, crystal structure, and electron density states of “fresh” (i.e. not used before) and used (i.e. one or more prior cycles) catalysts stayed the same as reflected by the results of SEM, TEM, XRD and XPS (FIGS. 10J, 11A-11D, 12A-12D, 13 and 14). All of these results showed the considerable stability of highly active Pt/GaN boundary.

TABLE 6
Reusability results for methane transformation over Pt1/GaN

| | | | Yield (μmol/g) | | | |
|---|---|---|---|---|---|---|
| | | Temperature | Cyclo- | | | |
| Entry | Catalyst | (° C.) | hexane | Benzene | Toluene | Xylene |
| 1 | Pt₁/GaN | 88.67 | 6.34 | 0.81 | n.d. | n.d. |
| 2 | Pt₁/GaN | 86.13 | 6.52 | 1.05 | n.d. | n.d. |
| 3 | Pt₁/GaN | 88.00 | 6.53 | 0.89 | n.d. | n.d. |
| 4 | Pt₁/GaN | 87.02 | 6.30 | 0.94 | n.d. | n.d. |
| 5 | Pt₁/GaN | 87.41 | 6.39 | 0.92 | n.d. | n.d. |

n.d. = not detectable

In summary, a methane liquification via a selective generation of cyclohexane was achieved, catalyzed the Pt/GaN catalysts having Pt clusters supported on GaN. It was demonstrated that the present method converts methane gas into liquid cyclohexane and “green hydrogen” with high selectivity (e.g. up to 92%) and productivity (41 μmol g⁻¹). Homogeneously dispersed Pt clusters on GaN are essential to achieve methane activation and the subsequent benzene hydrogenation via surface-hydrogen-transfer (SHT) process with the surface bound H-atoms for efficient production of cyclohexane. The SHT occurs at the heterojunction boundary of electron-rich platinum cluster (Pt) loaded on the methane-activating gallium nitride (GaN) host. The catalyst system was found to be stable and can be reused multiple times without diminishing the catalytic activity of the catalyst. Since the catalyst was immobilized on the flask-wall in the present Examples, the method can be readily adapted into flow systems within existing facilities for large scale applications. Moreover, the present method provides the advantage of a concurrent generation of “green hydrogen” as a side product while producing the high-valued cyclohexane.

The scope is indicated by the appended claims.

1. A catalyst for the conversion of methane to cyclohexane, the catalyst comprising:
gallium nitride, zinc oxide, gallium oxide or a combination thereof; and
platinum clusters deposited at the surface of the gallium nitride, the zinc oxide, the gallium oxide or the combination thereof, wherein the platinum clusters collectively represent from about 0.75 to about 4% by weight of the catalyst.

2. The catalyst of claim 1, wherein the catalyst comprises at least 95% by weight of gallium nitride.

3. The catalyst of claim 1, wherein the platinum clusters collectively are from about 0.75 to about 2% by weight of the catalyst.

4. The catalyst of claim 1, wherein the catalyst is a nanoparticle.

5. The catalyst of claim 2, wherein the catalyst has a platinum 4f_{7/2} binding energy peak of less than 74.0 eV determined by X-ray photoelectron spectroscopy.

6. The catalyst of claim 2, wherein the catalyst has a platinum 4f_{5/2} binding energy peak of less than 71.0 eV determined by X-ray photoelectron spectroscopy.

7. A method of producing cyclohexane comprising the step of:
contacting the catalyst as defined in claim 1 and methane in a reaction vessel at a temperature of from about 250° C. to about 350° C. for a time sufficient to produce the cyclohexane.

8. The method of claim 7, wherein the time sufficient to produce the cyclohexane is between 1 to 3 hours.

9. The method of claim 7, wherein the step of contacting is performed under vacuum or in an inert atmosphere.

10. The method of claim 7, further comprising drying the methane before the step of contacting the methane with the catalyst.

11. The method of claim 7, further comprising cleaning the catalyst before the step of contacting the methane with the catalyst.

12. The method of 11, wherein cleaning the catalyst comprises purging the catalyst with methane.

13. A process of making the catalyst as defined in claim 1, the process comprising the step of:
depositing from about 0.75 to about 4% by weight of platinum on the surface of the gallium nitride, the zinc oxide, the gallium oxide or the combination thereof to obtain the catalyst by chemical reduction.

14. The process of claim 13, wherein the chemical reduction is a photodeposition.

15. The process of claim 13, wherein the gallium nitride is a GaN nanoparticle.

16. The process of claim 13, further comprising washing the catalyst after the step of depositing.

17. The process of claim 16, further comprising drying the catalyst after the step of washing.
